(19) United States
(12) Patent Application Publication
(10) Pub. No.: US 2007/0083075 A1
(43) Pub. Date: Apr. 12, 2007

(54) Method and apparatus for applying energy to tissues

(76) Inventor: Moshe Ein-Gal, Ramat Hasharon (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/247,172

(22) Filed: Oct. 12, 2005

(51) Int. Cl. A61N 1/00; A61N 2/00; A61B 17/52

(52) U.S. Cl. 600/13; 600/9

(57) ABSTRACT

Apparatus including first and second pairs of electromagnetic energy elements connected to a source of electromagnetic energy and to a controller, the first and second pairs of electromagnetic energy elements being arranged such that an electromagnetic field of the first pair deliberately overlaps an electromagnetic field of the second pair, wherein the controller energizes the first pair to create a first electromagnetic field sufficient to increase an electrical resistance of tissue and create a zone of increased electrical resistance that lies within the electromagnetic field of the second pair, and wherein the controller then energizes the second pair to create a second electromagnetic field such that flux lines of the second electromagnetic field are different than they would have been if the zone of increased electrical resistance had not been created.

### FIELD OF THE INVENTION

The present invention relates generally to methods and apparatus for applying energy (e.g., RF energy) to tissues, e.g., for tissue coagulation.

### BACKGROUND OF THE INVENTION

It is well known in the prior art to apply energy from electrodes to tissues, for various purposes, such as but not limited to, ablation, coagulation, necrosis, etc. For example, RF energy may be applied to pairs of small electrodes in a bipolar mode of operation, wherein one of the electrodes is the cathode and the other is the anode. Electromagnetic energy is deposited between the electrodes, wherein in simplistic terms, field lines form a sort of ellipsoid-like envelope around the electrodes. If the deposited energy is sufficient to cause tissue coagulation or necrosis, the coagulated tissue electrical resistance increases and, consequently, current and energy deposition in that tissue is significantly reduced.

### SUMMARY OF THE INVENTION

The present invention seeks to provide novel methods and apparatus for applying energy to tissues, as is described more in detail hereinbelow.

In accordance with a non-limiting embodiment of the invention, pairs of electrodes are arranged such that the electromagnetic field created by one pair is deliberately in a space shared by the electromagnetic field of the other pair. The first pair of electrodes may be energized to create an electromagnetic field that causes coagulation or necrosis of shared tissue. The associated electrical resistance increase affects current flow in the shared tissue when the second electrode pair is energized. The electrical flux lines of the second pair are now altered due to the presence of the shared zone of increased electrical resistance. That is, electrical current path produced by the second pair is now different than the path taken if the zone of increased electrical resistance had not been created. This technique can be used to ablate, coagulate or otherwise treat tissue in manners and patterns heretofore not possible.

There is thus provided in accordance with an embodiment of the present invention apparatus including first and second pairs of electromagnetic energy elements connected to a source of electromagnetic energy and to a controller, the first and second pairs of electromagnetic energy elements being arranged such that an electromagnetic field of the first pair deliberately overlaps an electromagnetic field of the second pair, wherein the controller energizes the first pair to create a first electromagnetic field sufficient to increase an electrical resistance of tissue and create a zone of increased electrical resistance that lies within the electromagnetic field of the second pair, and wherein the controller then energizes the second pair to create a second electromagnetic field such that flux lines of the second electromagnetic field are different than they would have been if the zone of increased electrical resistance had not been created. In accordance with an embodiment of the present invention, energy due to the second electromagnetic field is deposited in a larger volume compared to the volume where equal energy would be deposited had the zone of increased electrical resistance not been created.

The apparatus may further include an electrical resistance sensor adjacent at least one of the electromagnetic energy elements, the electrical resistance sensor being in communication with the controller, wherein the controller may control energy to the first pair and the second pair upon the electrical resistance sensor sensing a predetermined electrical resistance.

Similarly, the apparatus may further include a temperature sensor being in communication with the controller, wherein the controller may control energy to the first pair and the second pair upon the temperature sensor sensing a predetermined temperature.

Similarly, the controller may control energy to the first pair and the second pairs upon measuring a predetermined elapsed time period.

The electromagnetic energy elements may include, without limitation, RF electrodes or electromagnetic coils. In particular, the electrodes or the coils may be constructed with a collinear and/or concentric geometry.

There is also provided in accordance with an embodiment of the present invention a method including providing first and second pairs of electromagnetic energy elements connected to a source of electromagnetic energy, the first and second pairs of electromagnetic energy elements being arranged such that an electromagnetic field of the first pair deliberately overlaps an electromagnetic field of the second pair, energizing the first pair to deposit sufficient electromagnetic energy to increase an electrical resistance of tissue and create a zone of increased electrical resistance that lies within the electromagnetic field of the second pair, and energizing the second pair to create a second electromagnetic field such that flux lines of the second electromagnetic field are different than they would have been if the zone of increased electrical resistance had not been created.

### BRIEF DESCRIPTION OF THE DRAWINGS

The present invention will be understood and appreciated more fully from the following detailed description, taken in conjunction with the drawings in which:

FIG. 1 is a simplified illustration of first and second electrode pairs, arranged such that their electromagnetic fields are deliberately in each other's path, in accordance with an embodiment of the present invention, showing normal, undisturbed flux lines of each electrode pair;

FIG. 2 is a simplified illustration of energizing the first electrode pair before the other pair, in accordance with an embodiment of the present invention, thereby causing a zone of tissue with increased electrical resistance, that is, an electrical insulation zone;

FIG. 3 is a simplified illustration of energizing the second electrode pair whose electrical flux lines are now altered due to the presence of the electrical insulation zone, in accordance with an embodiment of the present invention;

FIG. 4 is a simplified illustration of a coaxial coil assembly, constructed and operative in accordance with an embodiment of the present invention, wherein the electromagnetic field of an inner coil is deliberately in the path of the electromagnetic field of an outer coil; and

FIG. 5 is a simplified illustration of an electrode array constructed and operative in accordance with another embodiment of the present invention, wherein pairs of electrodes are arranged such that the electromagnetic field of one pair is deliberately in the path of the electromagnetic field of another pair.

### DETAILED DESCRIPTION OF EMBODIMENTS

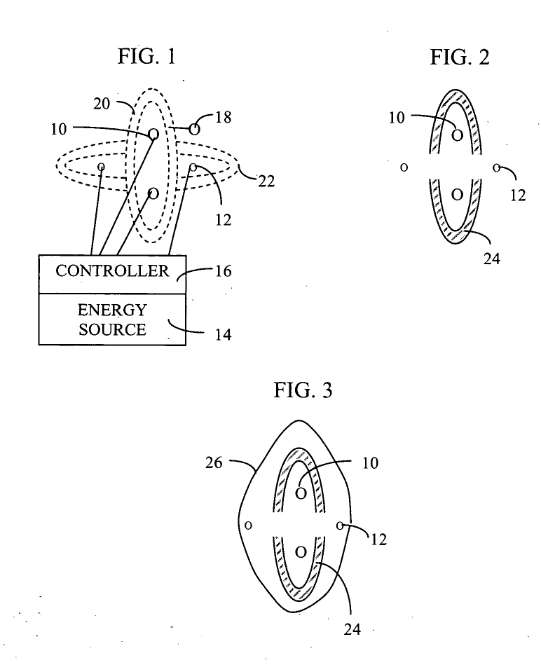

Reference is now made to FIG. 1, which illustrates apparatus for applying energy to tissues, constructed and operative in accordance with an embodiment of the present invention.

In accordance with a non-limiting embodiment of the invention, the apparatus may include first and second pairs of electromagnetic energy elements 10 and 12 connected to a source of electromagnetic energy 14 (e.g., an RF generator) and to a controller 16. The electromagnetic energy elements 10 and 12 may include RF electrodes in a bipolar mode of operation, wherein the electrodes alternate between being the cathode and anode, for example. The first and second pairs of electromagnetic energy elements 10 and 12 may be arranged such that an electromagnetic field of the first pair 10 deliberately overlaps an electromagnetic field of the second pair 12.

The apparatus may further include a sensor 18, which without limitation, may be an electrical resistance sensor (e.g., resistive element in electrical connection with tissue) or a temperature sensor (e.g., thermistor or thermocouple) adjacent at least one of the electromagnetic energy elements 10 or 12. As will be described below, sensor 18 may be used in conjunction with controller 16 to control the sequential operation of the first and second pairs of electromagnetic energy elements 10 and 12. (For the sake of simplicity and clarity, energy source 14, controller 16 and sensor 18 are omitted from FIGS. 2 and 3.) Electromagnetic energy elements 10 or 12 are characterized by electromagnetic fields 20 and 22, respectively. The electromagnetic fields 20 and 22 are shown in FIG. 1 as if there were no interference or disturbance between the two fields.

Reference is now made additionally to FIG. 2. In accordance with a non-limiting method of the invention, controller 16 energizes the first pair of electromagnetic energy elements 10 to create a first electromagnetic field 20 sufficient to increase an electrical resistance of tissue and create a zone of increased electrical resistance 24 in the tissue that lies within the electromagnetic field of the second pair electromagnetic energy elements 12. Referring to FIG. 3, controller 16 then energizes the second pair of electromagnetic energy elements 12 to create a second electromagnetic field 26. It is readily seen by comparison of FIGS. 1 and 3 that flux lines of the second electromagnetic field 26 are different than electromagnetic field 22, i.e., the lines are different they would have been if the zone of increased electrical resistance 24 had not been created. The second electromagnetic field 26 may extend over a greater volume than it would have extended if the zone of increased electrical resistance 24 had not been created (that is, second electromagnetic field 26 is greater in volume than electromagnetic field 22).

Controller 16 may control energy to the first and second pairs of electromagnetic energy elements 10 and 12 in a control loop with sensor 18. For example, controller 16 may shut off energy to the first pair of electromagnetic energy elements 10 and commence energizing the second pair of electromagnetic energy elements 12 upon the sensor 18 (e.g., electrical resistance or temperature sensor) sensing a predetermined parameter (e.g., electrical resistance or temperature). This predetermined parameter may be selected or calibrated to correspond to a desired level or degree of coagulation or necrosis, for example. Additionally or alternatively, controller 16 may control energy to the first and second pairs of electromagnetic energy elements 10 and 12 upon measuring a predetermined elapsed time period.

In the non-limiting embodiment illustrated in FIGS. 1-3, the electromagnetic energy elements may include RF electrodes.

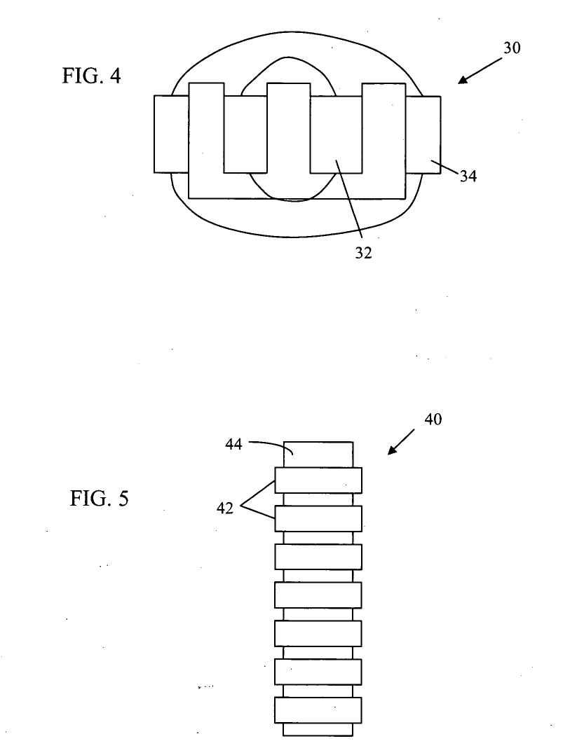

Reference is now made to FIG. 4, which illustrates a coaxial coil assembly 30, constructed and operative in accordance with an embodiment of the present invention. The coil assembly 30 may include an inner coil 32 and an outer coil 34, which may be concentric with inner coil 32. The electromagnetic field of inner coil 32 is deliberately in the path of the electromagnetic field of outer coil 34. The inner and outer coils 32 and 34 may be connected to a controller (like controller 16) and operated as described above to create different electromagnetic fields.

Reference is now made to FIG. 5, which illustrates an electrode array 40 constructed and operative in accordance with another embodiment of the present invention. Electrode array 40 may include pairs of annular electrodes 42 disposed on a shaft 44. The electrodes 42 may be arranged as bipolar pairs, such that the electromagnetic field of one pair is deliberately in the path of the electromagnetic field of another pair, the pairs being operated as described hereinabove. It is appreciated that the invention is not limited to the electromagnetic energy elements shown in the figures and other types and arrangements of electromagnetic energy elements are within the scope of the invention.

The scope of the present invention includes both combinations and subcombinations of the features described hereinabove as well as modifications and variations thereof which would occur to a person of skill in the art upon reading the foregoing description and which are not in the prior art.

What is claimed is:

1. Apparatus comprising:
first and second pairs of electromagnetic energy elements connected to a source of electromagnetic energy and to a controller, said first and second pairs of electromagnetic energy elements being arranged such that an electromagnetic field of said first pair deliberately overlaps an electromagnetic field of said second pair, wherein said controller energizes said first pair to create a first electromagnetic field sufficient to increase an electrical resistance of tissue and create a zone of increased electrical resistance that lies within the electromagnetic field of said second pair, and wherein said controller then energizes said second pair to create a second electromagnetic field such that flux lines of the second electromagnetic field are different than they would have been if the zone of increased electrical resistance had not been created.

2. Apparatus according to claim 1, wherein energy due to said second electromagnetic field is deposited in a larger volume compared to a volume where equal energy would be deposited without said zone of increased electrical resistance.

3. Apparatus according to claim 1, further comprising an electrical resistance sensor, said electrical resistance sensor being in communication with said controller, wherein said controller controls energy to said first pair and said second pair upon said electrical resistance sensor sensing a predetermined electrical resistance.

4. Apparatus according to claim 1, further comprising a temperature sensor in communication with said controller, wherein said controller controls energy to said first and second pairs of electromagnetic energy elements upon said temperature sensor sensing a predetermined temperature.

5. Apparatus according to claim 1, wherein said electromagnetic energy elements comprise RF electrodes.

6. Apparatus according to claim 1, wherein said electromagnetic energy elements comprise concentric and coaxial electromagnetic coils.

7. Apparatus according to claim 1, wherein said controller controls energy to said first and second pairs of electromagnetic energy elements upon measuring a predetermined elapsed time period.

8. A method comprising:
providing first and second pairs of electromagnetic energy elements connected to a source of electromagnetic energy, said first and second pairs of electromagnetic energy elements being arranged such that an electromagnetic field of said first pair deliberately overlaps an electromagnetic field of said second pair;
energizing said first pair to create a first electromagnetic field sufficient to increase an electrical resistance of tissue and create a zone of increased electrical resistance that lies within the electromagnetic field of said second pair; and
energizing said second pair to create a second electromagnetic field such that flux lines of the second electromagnetic field are different than they would have been if the zone of increased electrical resistance had not been created.

9. The method according to claim 8, wherein energy due to said second electromagnetic field is deposited in a larger volume compared to a volume where equal energy would be deposited without said zone of increased electrical resistance.

10. The method according to claim 8, further comprising sensing an electrical resistance adjacent at least one of said electromagnetic energy elements, and shutting off energy to said first pair and commencing energizing said second pair upon sensing a predetermined electrical resistance.

11. The method according to claim 8, further comprising sensing a temperature adjacent at least one of said electromagnetic energy elements, and shutting off energy to said first pair and commencing energizing said second pair upon sensing a predetermined temperature.
